(19) United States
(12) Patent Application Publication
(10) Pub. No.: US 2008/0221385 A1
(43) Pub. Date: Sep. 11, 2008

(54) HYPERTHERMIA TREATMENT DEVICE WITH X-RAY COMPUTED TOMOGRAPHY IMAGING

(76) Inventor: Peter Aulbach, Forchheim-Kersbach (DE)

Correspondence Address: SCHIFF HARDIN, LLP;PATENT DEPARTMENT, 6600 SEARS TOWER, CHICAGO, IL, 60606-6473, US

(21) Appl. No.: 11/853,886

(22) Filed: Sep. 12, 2007

(30) Foreign Application Priority Data
Sep. 12, 2006 (DE) 10 2006 042 730.0

(51) Int. Cl. A61N 1/40; A61N 2/04; A61B 6/00; H05B 6/02

(52) U.S. Cl. 600/10; 128/899; 600/12; 607/103; 378/21

(57) ABSTRACT

A medical technology device has an x-ray CT imaging system and a magnet system for generation of a radio-frequency alternating magnetic field for treatment of a tumor injected with ferrite nanoparticles, the x-ray CT system and the magnet system being combined to form a unitary structure. Imaging and tumor therapy can be conducted with one apparatus, and the monitoring of the course of the tumor therapy is simplified.

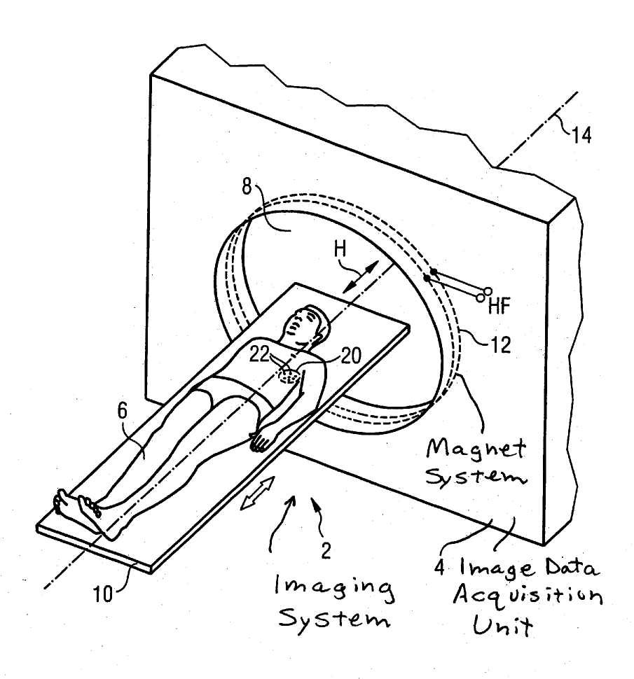

### BACKGROUND OF THE INVENTION

1. Field of the Invention

The invention concerns a medical technology device, and in particular a hyperthermia treatment device.

2. Description of the Prior Art

Medical technology devices support the physician both in diagnostics and in the implementation of therapeutic methods. In particular, imaging systems that serve not only as aids for pure diagnostics but also that allow the course of a therapeutic procedure to be detected (monitored) represent a significant tool in the planning and implementation of therapeutic procedures.

X-ray computed tomography (CT) and positron emission tomography (PET) represent particularly suitable modalities for the planning of a tumor therapy as well as for monitoring the course of the implemented therapeutic procedures. In addition to its high spatial resolution and the possibility to assess whether tumor tissue has already diffused into bones or not, a significant advantage of a conventional x-ray CT modality is the fact that such systems are widely available in oncology clinics. Moreover, it is advantageous that a good soft tissue contrast can be achieved and an efficient monitoring of the lesion after a procedure (i.e. the course of the procedure) is enabled with x-ray CT. Positron emission tomography combined with (x-ray) computed tomography (PET/CT) has turned out to be particularly advantageous for the course monitoring in the therapy of tumors. The metabolic information about the bioactivity that is acquired via PET is added to the morphological information of CT.

However, as before the actual therapeutic procedure represents a particular problem in the treatment of tumors, even when it is implemented in a minimally-invasive manner with thermal methods as is the case, for example, in a radio-frequency ablation (RFA) and laser-induced thermo-therapy (LITT). The placement of the electrodes or the placement of the optical conductor in the lesion, which ensues without the contrasted lesion being visible in the CT image, represents a particular problem in these methods. Moreover, during the procedure the risk exists of a tumor metastasis or a tumor cell dissemination causing local or regional relapses, that an occur in areas in which large vessels run through the lesion that lead to a local cooling and reduce the effect of the procedure.

Moreover, to monitor the course of a procedure it is always necessary to interrupt the procedure and to conduct a CT or PET examination on the patient in order to reinitiate and continue the procedure after the examination, requiring detachment and re-attachment of the electrodes or the optical conductors in the lesion. This causes significant time losses as well as risks for the patient.

### SUMMARY OF THE INVENTION

An object of the present invention is to provide a medical technology device with which a tumor treatment and the measures necessary for monitoring of its course can be implemented with optimally small time loss and simultaneously with optimally low risk for the patient.

This object is achieved according to the invention by a medical technology device having a unitary x-ray CT imaging system and magnetic system for generation of a radio-frequency alternating magnetic field for the treatment of a tumor injected with ferrite nanoparticles.

By the combination of the CT imaging system and the magnet system in one apparatus it is possible to track the course of a tumor treatment without problems between individual treatment segments, and the medical workflows required in the prior art are simplified and temporally shortened.

The invention is based on the insight that the therapy system already tested in tumor therapy in the Charité in Berlin (http://www.wdr.de/tv/qv/q21/110.0.phtml) is particularly suitable for a combination with a CT imaging system since an invasive procedure on the patient is not required with this therapy method and the magnet system can be arranged without any problems in proximity to the apparatus components required for the image acquisition. In this known therapy method, ferrite nanoparticles doped with a sugar (fluorodesoxyglucose (FDG)) are injected into the bloodstream of the patient in proximity to the tumor. The ferrite nanoparticles are iron oxide particles in the size of 10 to 15 nm. These ferrite nanoparticles thereby arrive via the arterial system at the active tumor regions in which they increasingly accumulate. The sugar is metabolized by the tumor cells, the released nanoparticles diffuse into the cell membranes of the tumor cells and increasingly accumulate there since the active tumor cells exhibit a high metabolic activity relative to healthy tissue. The patient is subsequently exposed to a radio-frequency alternating magnetic field. The oscillatory movement of the nanoparticles brought about by the alternating magnetic field leads to a heating and thermal destruction of the active tumor tissue. After conclusion of the procedure, the ferrite nanoparticles reside in the body of the patient until the body rejects the necrotic tumor tissue. A significant advantage of this method also is that even small metastases located far from the diagnosed tumor (remote metastases) are included in the treatment.

In a preferred embodiment of the invention, the CT imaging system has a scanning unit for generation of a slice image from a body region of a patient, which imaging system exhibits a central opening for insertion of a patient positioning table, and in which imaging system is arranged a magnetic coil arranged around the opening, the coil axis of which magnetic coil runs parallel to the central axis of the opening. In this way the generation of a slice image from the body region comprising the tumor can ensue practically immediately at the conclusion of a procedure. In this manner the procedure can be temporally sub-divided into individual segments without problems so that a damage of healthy tissue located in the surroundings of the tumor, which damage goes beyond the destruction of the tumor, can be efficiently avoided.

Such an arrangement can be integrated particularly simply into the imaging system as an exclusively x-ray CT system, or as an imaging system in which an x-ray CT system is combined with a PET system. A further particular advantage of such a combination also is that the ferrite nanoparticles simultaneously represent an excellent contrast agent for x-ray image generation that is harmless in terms of health considerations and has a relatively long residence duration in the body of the patient, which in practice lasts until the end of the procedure and up to the point in time in which the therapeutic success occurs, namely a rejection of the necrotic tumor tissue.

### BRIEF DESCRIPTION OF THE DRAWINGS

The single FIGURE schematically illustrates an exemplary embodiment of a medical technology device according to the invention.

### DESCRIPTION OF THE PREFERRED EMBODIMENTS

According to the FIGURE, a medical technology device has comprises an imaging system 2 (namely an x-ray CT system or a combination of an x-ray CT system and a PET system) with an image data acquisition unit 4 (a gantry) with x-ray tube/detector system (CT) arranged such that it can pivot, or a detector system (PET) arranged such that it can pivot, for generation of a slice image of a body region of a patient 6. The image data acquisition unit 4 has a central opening 8 for insertion of a patient positioning table 10 on which the patient 6 is borne. A magnet system 12 for generation of a radio-frequency alternating magnetic field H (in this example a magnetic coil arranged around the opening 8 and connected with a high voltage source HF, with the coil axis of the magnetic coil being parallel to the center axis 14 of the opening 8) is arranged around the opening 8 within the image data acquisition unit 4, forming a unitary structure.

The patient 6 exhibits a tumor 20 in which previously injected ferrite nanoparticles 22 have accumulated. For therapeutic treatment, the patient 6 is inserted into the opening 8 so that the tumor 20 is located in the effective region of the magnet system 12, i.e. in a region of high magnetic field strength. A therapeutic treatment of the tumor 20 ensues by activation of the magnet system 12. After conclusion of the treatment a series of slice images is acquired from the region of the tumor 20 by displacement (shifting) of the patient positioning table 10 and this series is assembled into a 3D image and evaluated for further therapy planning. If necessary, the treatment can subsequently be immediately continued. A time-consuming removal and relocation of the patient 6 from between the therapy location and a diagnosis location is thus avoided.

Although modifications and changes may be suggested by those skilled in the art, it is the intention of the inventor to embody within the patent warranted hereon all changes and modifications as reasonably and properly come within the scope of his contribution to the art.

I claim as my invention:

1. A medical technology device comprising:
a magnet system, configured to interact with a patient, which generates a radio-frequency alternating magnetic field directed at a tumor in the patient injected with ferrite nanoparticles;
an imaging system including an x-ray CT system that generates an image of a region of the subject in which said tumor is situated; and
said magnet system and said imaging system being combined to form a unitary structure configured to receive the patient therein for both treatment with said magnet system and imaging with said imaging system.

2. A medical technology device as claimed in claim 1 wherein said imaging system also comprises a PET system.

3. A medical technology device as claimed in claim 1 wherein said imaging system comprises an acquisition unit that acquires data representing slice images in said region of the patient, said imaging system having a central opening and comprising a patient positioning table, configured to receive the patient thereon, that is movable through said central opening, and wherein said magnet system comprises a magnetic coil disposed around said opening, said magnetic coil having a coil axis proceeding parallel to a center axis of said opening.

4. A medical technology device as claimed in claim 1 wherein said ferrite nanoparticles have a size in a range between 10 nm and 15 nm.

5. A medical technology device as claimed in claim 1 wherein said ferrite nanoparticles comprise nanoparticles doped with a sugar.

6. A medical technology device as claimed in claim 5 wherein said sugar is fluorodesoxyglucose.
